(19) United States
(12) Patent Application Publication
(10) Pub. No.: US 2003/0153951 A1
(43) Pub. Date: Aug. 14, 2003

(54) Methods and devices for treating arrhythmias using defibrillation shocks

(76) Inventors: Raymond E. Ideker, Birmingham, AL (US); Nipon Chattipakorn, Birmingham, AL (US)

Correspondence Address: MYERS BIGEL SIBLEY & SAJOVEC, PO BOX 37428, RALEIGH, NC, 27627, US

(21) Appl. No.: 10/071,269

(22) Filed: Feb. 8, 2002

(51) Int. Cl. A61N 1/362

(52) U.S. Cl. 607/3

(57) ABSTRACT

The present invention includes both methods and apparatus for improving defibrillation. In particular, the present invention includes methods and devices for administering a calcium channel blocker and/or calmodulin kinase inhibitor that prevents afterdepolarizations and antiarrhythmic drugs to decrease the threshold shock from a therapeutic threshold shock.

This invention was made with Government support under National Institutes of Health research grant HL-42760. The Government may have certain rights to this invention.

### FIELD OF THE INVENTION

The present invention relates to methods and apparatus for implantable medical devices, such as cardiac pacemakers, defibrillators and any of a variety of drug delivery systems. More particularly, the present invention relates to methods and apparatus used to reduce shock strength.

### BACKGROUND OF THE INVENTION

Electrical defibrillation has been routinely practiced to terminate ventricular fibrillation (VF) for decades. However, the mechanism of defibrillation is still unclear. Despite the wide use of external and internal defibrillators that already save thousands of lives, there remains a need to improve defibrillation efficacy. Understanding the basic mechanism of defibrillation is an important step toward this goal. For example, cardiac mapping studies of defibrillation have demonstrated that for shocks of the strength near the defibrillation threshold (DFT), the immediate post-shock activation arose from the weakest shock field region. See, e.g., Chattipakorn et al, “Prediction of defibrillation outcome by epicardial activation patterns following shocks near the defibrillation threshold”, J. Cardiovasc. Electrophysiol, 11: 1014-1021 (2000); Usui et al., “Epicardial sock mapping following monophasic and biphasic shocks of equal voltage with an endocardial lead system”, J. Cardiovasc. Electrophysiol, 7: 322-334 (1996). These findings have led to the development of devices to treat fibrillation. See, e.g., KenKnight et al., “Marked reduction of ventricular defibrillation threshold by application of an auxiliary shock to a catheter electrode in the left posterior coronary vein of dogs”, J Cardiovasc Electrophysiol, 11: 900-906 (2000); Meisel et al., “Transvenous biventricular defibrillation”, Am J Cardiol 86: K76-K85 (2000); and Roberts et al., “The middle cardiac vein—a novel pathway to reduce the defibrillation threshold”, J Interv Card Electrophysiol 3: 55-60 (1999).

Previously, it was believed that reentry was the mechanism responsible for failed defibrillation. However, this concept has been challenged by results from both optical and electrical mapping defibrillation studies that used only shocks having a strength at or above the defibrillation threshold. These shock strengths that have been used clinically to terminate ventricular fibrillation. See, e.g., Chattipakorn et al. (2000), supra and Usui et al., supra. Results from those studies have consistently demonstrated that following near defibrillation threshold shocks that failed to defibrillate, the first several post-shock activation cycles arose focally, rapidly and repeatedly from a region where the shock field is weakest. Id. These activation cycles are then propagated across the heart in an organized pattern for several cycles before degenerating into ventricular fibrillation. Although the cause of these post-shock focal activations is not known, delayed afterdepolarizations (DADs) have been suggested as a possible mechanism for these rapid post-shock activations. See, e.g., Chattipakorn et al., supra; Ideker et al., “Defibrillation Mechanisms: The Parable of the Blind Men and the Elephant?”, J. Cardiovasc. Electrophysiol, 11: 1008-1013 (2000); and Li et al., “Defibrillation shocks produce different effects on Purkinje fibers and ventricular muscle: implications for successful defibrillation, refibrillation and postshock arrhythmia”, J. Am. Coll. Cardiol. 22: 607-614 (1993). It is believed that this proposition has not been verified and has been ignored due to the belief that post-shock reentry is the sole mechanism responsible for failed defibrillation.

A delayed afterdepolarization has been proposed as a possible mechanism responsible for post-shock activations according to consistent findings from both optical and electrical mapping studies which showed that, following near defibrillation threshold shocks that fail to defibrillate, rapid focal activations always arose rapidly and repetitively after the shock before degenerating into ventricular fibrillation. Yet, reentry was never observed during these post-shock cycles as confirmed by a recent report using a three-dimensional mapping technique. See, Chattipakorn et al., “Origin of the earliest activation after ventricular defibrillation: Insight from a 3-dimensional cardiac mapping”, Pacing and Clinical Electrophysiology, 24: 669 (2001); and Chattipakorn et al., “Three-dimension cardiac mapping of the earliest activation following upper limit of vulnerability shocks”, Pacing and Clinical Electrophysiology, 24: 561 (2001). Although delayed afterdepolarization has been hypothesized as a possible mechanism responsible for these focal activation cycles, this hypothesis has never been verified. See, e.g., Shibata et al., “Epicardial activation following unsuccessful defibrillation shocks in dogs” Am. J. Physiol, 255: H902-H909 (1988); Chattipakorn et al., “Mechanism of Defibrillation” In Fighting Sudden Cardiac Death: A Worldwide Challenge, Futura Publishing Co., pp. 593-615 (2000); Li et al., “Defibrillation shocks produce different effects on Purkinje fibers and ventricular muscle: implications for successful defibrillation, refibrillation and postshock arrhythmia”, J. Am. Coll. Cardiol., 22: 607-614 (1993); and Ideker et al.

Objects used in improving defibrillation include numerous medical devices. Many of these medical devices have electrical circuit components and are well known in medical science. Some of the most common forms of such implantable devices are pacemakers and defibrillators.

A pacemaker is an implantable medical device which generates electrical pulses to an electrode implanted adjacent to the patient's heart in order to stimulate the heart so that it can beat at a desired rate. A normal human heart contains a natural pacemaker which develops a normal rhythmic electrical excitation. The human heart normally maintains its own well-ordered intrinsic rhythm through generation of stimuli by pacemaker tissue that results in a wave of depolarization that spreads through specialized conducting tissue and then into and through the myocardium. In a normal functioning heart, various physiological regulatory mechanisms cause the heart to beat at a rate that maintains cardiac output at a sufficient level to meet the metabolic needs of the body. Abnormalities of cardiac tissue can lead to abnormalities of heart rhythm. These abnormalities are known as arrhythmias. Arrhythmias generally stem from one of two causes: namely, abnormalities of impulse generation and/or abnormalities of impulse propagation. When the heart malfunctions, due to age or disease, an implantable pacemaker often is used to stimulate the heart properly. Pacemakers have been developed that can automatically change the rate at which the pacemaker provides stimulating pulses to the heart in response to a sensed physiological parameter. The physiological parameter provides information that indicates whether the heart rate should increase or decrease, as dictated by the physiological needs of the subject. U.S. Pat. No. 4,830,006 provides a further description of cardiac physiology and the theory of pacemaker operation, the contents of which are hereby incorporated by reference.

Similarly, implantable defibrillators can be configured to sense physiological parameters in order to determine when to supply a defibrillating shock to a patient's heart. Ventricular fibrillation is a condition characterized by rapid, chaotic electrical and mechanical activity of the heart's excitable myocardial tissue, and results in an almost instantaneous cessation of blood flow from the heart due to the uncoordinated or ineffectual action of the ventricles. Defibrillation is a technique employed to terminate fibrillation by applying one or more high energy electrical pulses to the heart in an effort to overwhelm the contractions of individual tissue sections and to restore the synchronized contraction of the total mass of tissue. The defibrillation shocks can be delivered while the patient is conscious.

Drug therapy has been found to be effective in preventing the development of arrhythmias and in restoring normal heart rhythms once an arrhythmia has occurred. One such drug, flunarizine, a calcium channel blocker, has illustrated the ability to effectively terminate arrhythmias due to delayed afterdepolarizations and to prevent their reinduction. See, Vos et al., “The effect of an entrainment protocol on Ouabain-induced ventricular tachycardia”, Pacing and Clin. Electrophys, 12: 1485-1493 (1989); Vos et al., “Flunarizine allows differentiation between mechanisms of arrhythmias in the intact heart”, Circulation, 81: 343-349 (1990); Vos et al., “Termination of ouabain-induced ventricular tachycardia by flunarizine in conscious dogs”, Eur J Pharmacol, 165: 139-145 (1989); and Vos et al., “Further observations to confirm the arrhythmia mechanism-specific effects of flunarizine”, J Cardiovasc Pharmacol 19: 682-690 (1992). Due to flunarizine's effect on intracellular calcium, it can be used to differentiate between arrhythmias resulting from triggered activity and other mechanisms. Conventional drugs have been used to inhibit or prevent the occurrence of reentry after the defibrillation shock, by either prolonging the refractory period (class III antiarrhythmic drugs) or preventing the occurrence of new activation (class I drugs). It may be desirable to improve drug delivery systems through defibrillation efficacy. In light of the above, there remains a need to provide improved methods and devices for treating arrhythmias.

### SUMMARY OF THE INVENTION

The present invention includes methods, systems, devices and computer program products for treating arrhythmias that may reduce the shock strength of a defibrillation threshold shock. One aspect of the present invention is a method of decreasing the shock strength needed to treat an arrhythmia comprising detecting an arrhythmia in the heart of a subject; administering a therapeutic electric shock to the heart of the subject to treat the arrhythmia; and contemporaneously administering a pharmacological agent comprising of at least one of a calcium channel blocker, an antiarrhythmic drug, and/or a calmodulin blocker to the subject in an amount effective to decrease the strength of the shock required to treat the arrhythmia. This method may be used in either atrial or ventricular arrhythmias.

Another aspect of the present invention includes cardiac devices that are used to decrease the shock strength of a defibrillation threshold shock. These cardiac devices may be internal or external and include sensing means for determining cardiac cycles. These cardiac cycles include arrhythmias and fibrillations. The devices may be used to provide a defibrillation threshold shock while contemporaneously administering a calcium channel blocker, an antiarrhythmic drug or a calmodulin blocker to said subject. Alternatively, the calcium channel blocker, antiarrhythmic drug or calmodulin blocker may be administering outside the scope of the device when an arrhythmia is occurring while the device provides a therapeutic shock.

### BRIEF DESCRIPTION OF THE FIGURES

FIG. 1 is a schematic illustration of a subject with an implantable cardiac device in position;

FIG. 2 is a schematic illustration of an implantable cardiac device according to the embodiments of the present invention;

FIG. 3 is a schematic illustration of a cardiac device according to the embodiments of the present invention;

FIG. 4 is a schematic illustration of an implantable cardiac device according to other embodiments of the present invention;

FIG. 5 is a schematic illustration of operational circuitry according to embodiments of the present invention;

FIG. 6 is a block diagram of a method for controlling the delivery of defibrillation shocks according to the embodiments of the present invention.

FIG. 7 is a graph of the delivered (leading edge) voltage and the total energy for the defibrillation threshold;

FIG. 8 is a graph of the current, impedance, and systolic blood pressure in four measured groups after a flunarizine application.

### DETAILED DESCRIPTION OF EMBODIMENTS OF THE INVENTION

The present invention now will be described more fully hereinafter with reference to the accompanying specification and drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used in the description of the invention is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety.

“Contemporaneously” means existing, occurring, or originating during the same time period.

“Cycle” herein refers to a wave of activation propagating across the epicardium of the heart, which in the normal, healthy heart is associated with a single contraction of the atria and ventricles, or a single “beat” of the heart.

“Triggered activity” is single or repetitive firing of a myocardial cell or a group of cells caused by reexcitation (afterdepolarization which occurs after repolarization has begun). Repolarization is the electrochemical process of cell recovery after excitation of the heart muscle. There are two different kinds of afterdepolarization: (1) early, whose causes include: a) low potassium blood levels; b) slow heart rate; and c) drug toxicity (i.e., quinidine causing a torsades de pointes form of ventricular tachycardia; and (2) late/delayed, which occurs after the cell has repolarized. These may cause rapid firing or triggered activity due to: 1) premature beats; 2) increased calcium blood levels; 3) increased adrenaline levels; and 4) digitalis toxicity.

Embodiments of the present invention may include cardiac devices such as pacemakers, defibrillators and drug delivery systems. These devices may be used externally or internally. The present invention is intended primarily for use on human subjects, but may optionally be carried out on other subjects for veterinary purposes.

In certain embodiments, administering a pharmacological agent including at least one of a calcium channel blocker, an antiarrhythmia drug and/or a calmodulin blocker may prevent calcium overload and most likely inhibit a delayed afterdepolarization, thus reducing the defibrillation threshold. Calcium channel blockers include amiodarone, bepridil, D600, diltiazem, felodipine, flunarizine, isradipine, nicardipine, nifedipine, nimodipine, and verapamil. Flunarizine, in particular, has shown that it may reduce the defibrillation threshold. Other drugs such as calmodulin blockers and antiarrhythmic drugs may also reduce the defibrillation threshold. These drugs include adenosine, doxorubicin, ryanodine, ethmozin, aprindine, ibutilide, dofetilide, calmodulin blockers, and/or calmodulin kinase inhibitors.

It is known in the art that delayed afterdepolarization is caused by oscillations of the transmembrane potential that depend on the preceding action potential for their generation and can be provoked by the increase in activation rate. See, e.g., Antzelevitch et al., “Cardiac Arrhythmias: Reentry and Triggered Activity”, in Heart Physiology and Pathophysiology, Academic Press, pp. 1153-1179 (2001); and Waldo et al., “Mechanisms of Cardiac Arrhythmias and Conduction Disturbances”, in Hurst's The Heart, McGraw-Hill Company, pp. 751-796 (2001). It can give rise to new activation fronts, i.e. triggered activity, if they reach a critical threshold for new activations. During fibrillation, heart rate is greatly elevated. Following a defibrillation shock, additional factors such as increased sympathetic tone, myocardial stretch, tissue damage, and reperfusion may be involved. These factors alone or in an additive fashion help promote delayed afterdepolarization. In addition, recent optical mapping studies have demonstrated that following near-defibrillation threshold shocks, complete repolarization is observed followed by a 40-60 ms quiescent period after which repetitive focal activations appear on the epicardium and later degenerate into ventricular fibrillation. These findings suggest that the first ectopic cycle arises after complete repolarization is achieved after the shock.

Embodiments of the present invention suggest that for shocks of the strength at or above the defibrillation threshold, afterdepolarizations may be a mechanism responsible for failed shocks and pharmacological intervention to prevent afterdepolarizations could help to improve defibrillation efficacy. The present invention allows for the use of calcium channel blockers and/or calmodulin kinase inhibitors to improve defibrillation techniques. Additionally, there may be chemical substance that can block or stimulate intracellular signaling that may prevent calcium overload. The mechanism of how calcium channel blockers and/or calmodulin kinase inhibitors prevent the occurrence of delayed afterdepolarization is not completely clear. It is believed that calcium channel blockers and/or calmodulin kinase inhibitors may block the Na⁺/Ca⁺⁺ exchange. They may also influence the calcium release channel of the sarcoplasmic reticulum as well as blocking the transient inward current. The application of calcium channel blockers and/or calmodulin kinase inhibitors that prevent the occurrence of a delayed afterdepolarization could significantly improve defibrillation. Calcium channel blocking agents and/or calmodulin kinase inhibitors affect the movement of calcium into the cells of the heart and blood vessels. As a result, they relax blood vessels and increase the supply of blood and oxygen to the heart while reducing its workload. Some of the calcium channel blocking agents and/or calmodulin kinase inhibitors may be used to relieve and control angina pectoris (chest pain).

As noted above, along with calcium channel blockers, calmodulin blockers, calmodulin kinase inhibitor and antiarrhythmic drugs may prevent delayed afterdepolarizations. Once the delayed afterdepolarizations have been prevented, the next electrophysiological mechanism causing defibrillation shocks to fail is probably initiation of reentry by the shock. The induction and stability of reentry and its degeneration into fibrillation can probably be deceased by administering antiarrhythmic drugs that prolong the action such as ibutilide or dofetilide. Therefore, the administration of a drug that prevents delayed afterdepolarizations together with an antiarrhythmic drug that prolongs refractory periods and action potentials should lower the shock strength needed for defibrillation even more than either of the two drugs alone.

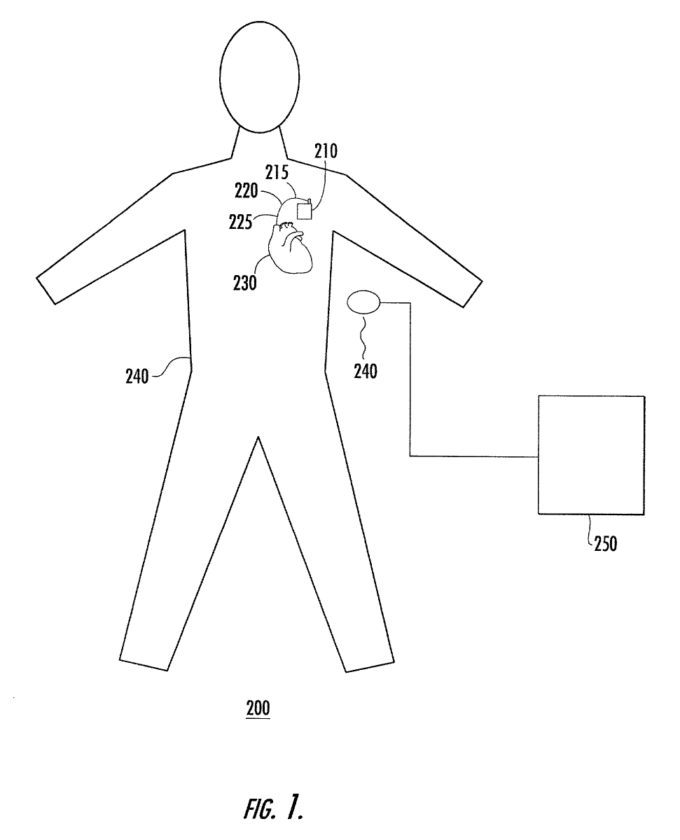

FIG. 1 is a schematic drawing illustrating generally, by way of example, but not by way of limitation, of certain embodiments of the present invention. FIG. 1 illustrates portions of a cardiac device 210 and an environment in which it is used. In FIG. 1, the overall system 200 includes an implantable cardiac device 210, also referred to as “an electronics unit”, which is coupled by a lead 220 to the heart 230 of patient 240. Lead 220 may also be referred to as a catheter, wire, cable or other connector means that may include one or more electrodes. The system 200 can include an external programmer 250 to provide for wireless communication with cardiac device 210 using a telemetry device 240. In one embodiment, external programmer 250 includes a visual or other display for providing information to a user regarding operation of implanted device 210. Lead 220 includes a proximal end 215, which is coupled to cardiac device 210, and a distal end 225, which is coupled to one or more portions of heart 230. The cardiac device may include sensing means for sensing an arrhythmia or a fibrillation. The cardiac system 210 may also include means for administering a therapeutic shock and means for delivering a drug.

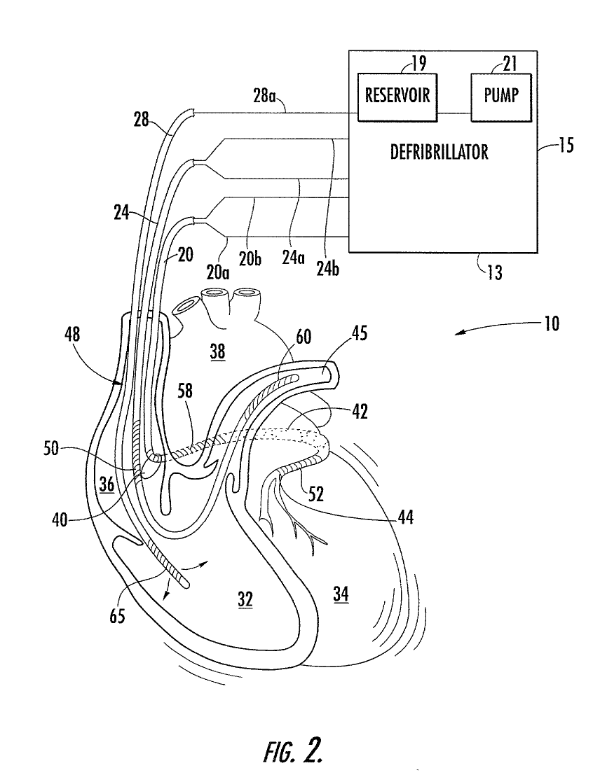

FIG. 2 depicts one embodiment of the present invention. In overview, FIG. 2 depicts an implantable system for the defibrillation of the atria of a patient's heart may comprise of (a) a first pair of defibrillation electrodes configured for delivering a first defibrillation pulse or a drug along a first current pathway in the heart; (b) a pulse generator operatively associated with the first pair of defibrillation electrodes for delivering the first defibrillation pulse or for delivering the drug to the bloodstream; (c) a second pair of defibrillation electrodes configured for delivering a second defibrillation pulse along a second current pathway in the heart, or for delivering the drug to the bloodstream, with the second current pathway different from the first current pathway; and (d) a pulse generator operatively associated with the second pair of defibrillation electrodes for sequentially delivering the second defibrillation pulse after the first defibrillation pulse. The term “electrode” as used herein is used interchangeably, with the terms “lead” and “catheter”. The electrode pairs may be placed in a variety of different locations, as long as different current pathways for the first and second pulse are thereby achieved. A single electrode may participate in more than one electrode pair, so that, for example, two current pathways are achieved through three defibrillation electrodes. Additional electrodes may be tied together to one member of an electrode pair to provide a single pole, if so desired, and additional electrodes may be provided for following the first and second shocks with additional shocks. Additionally, any of the electrodes may also be configured as a catheter that may release a drug such as a calcium channel blocker, a calmodulin blocker, a calmodulin kinase inhibitor and an antiarrhythmic drug. The catheter may also have a lumen for conveyance of fluids along its length and a hollow fixation means for delivering the fluids into the heart tissue or bloodstream. Additionally, the catheter may include a timing mechanism to allow for the slow or rapid elution of the calcium channel blocker, calmodulin blocker, calmodulin kinase inhibitor or antiarrhythmic drug.

In FIG. 2, the first pair of defibrillation electrodes comprises a defibrillation electrode that may be positioned in the right atrium or superior vena cava of the heart, and a defibrillation electrode positioned in the distal coronary sinus or great cardiac vein of the heart. The electrodes themselves may be configured for positioning in the indicated location as well as being configured in an ideal way to transport a calcium channel blocker or drug into the bloodstream. Numerous alternatives for the second pair of defibrillation electrodes forming a second pathway are possible. For example, the second pair of defibrillation electrodes may comprise: (A) a defibrillation electrode positioned in the proximal coronary sinus of the heart, and a defibrillation electrode positioned anterior to the left atrium of the heart (e.g., in the left pulmonary artery, or on the external surface of a device implanted subcutaneously in the left thoracic region of the patient); (B) a defibrillation electrode positioned in the left pulmonary artery of the heart, and a defibrillation electrode positioned in the right ventricle of the heart; (C) a defibrillation electrode positioned in the distal coronary sinus of the heart, and a defibrillation electrode positioned in the right ventricle of the heart; (D) a defibrillation electrode positioned in the left pulmonary artery of the heart, and a defibrillation electrode positioned in the right atrium of the heart; (E) a defibrillation electrode positioned in the left pulmonary artery of the heart, and a defibrillation electrode positioned in the distal coronary sinus of the heart (the electrode positioned in the distal coronary sinus may optionally be tied together with an electrode positioned in the right atrium as one pole); (F) a defibrillation electrode positioned in the proximal coronary sinus of the heart, and a defibrillation electrode positioned in the right atrium of the heart; or (G) a defibrillation electrode positioned in the proximal coronary sinus of the heart, and a defibrillation electrode positioned in the distal coronary sinus of the heart (the electrode positioned in the distal coronary sinus may optionally be tied together with an electrode positioned in the right atrium as one pole). Optionally, any of the above may also be considered as (A) a catheter made of permanently implantable materials, it has electrical continuity from end to end for sensing cardiac activity, it has a lumen for conveying fluidic agents along its length, and a hollow fixation means for delivering fluidic agents to a depth within the heart tissue; or (B) an acute catheter made of nonimplantable materials that may introduce a drug into the bloodstream.

Again, the electrodes may be configured for positioning in the indicated locations, and numerous variations on the foregoing will be readily apparent to those skilled in the art. For example, the first defibrillation pulse or drug delivery could be delivered by the second pair of electrodes indicated above, and the second defibrillation pulse or drug delivery could be delivered by the first pair of electrodes indicated above (in which case the indicated second pair of electrodes serves as the “first pair” and the indicated first pair serves as the “second pair”). In addition, multiple electrodes may be implanted to provide three, four, or five or more different alternative electrode pairs and current paths, and the electrode coupling to the pulse generator switched after implantation of the electrodes to optimize the electrode configuration or drug delivery for a particular patient.

FIG. 2 illustrates one embodiment of a preferred defibrillation system of the instant invention. The defibrillator 10 of FIG. 2 includes an implantable housing 13 which contains a hermetically sealed electronic circuit 15. The defibrillator also includes a first catheter 20, a second catheter 24, and a third catheter 28, all of which are insertable into the heart 30 without the need for surgical incision. Each of the catheters 20, 24, 28 contain electrode leads 20a, 20b, 24a, 28a, respectively for connecting at least one electrode to the electronic circuit 15 in the housing.

In the embodiment of FIG. 2, the implantable defibrillation system includes a first defibrillation electrode 50 configured for positioning in the right atrium (R-A) 36 or superior vena cava 48. Also illustrated in sectional view is the interior of the right ventricle (RV) 32, along with the exterior of the left atrium (LA) 38 and the exterior of the left ventricle (LV) 34. A second defibrillation electrode 52 is configured for positioning in the distal coronary sinus (DCS) 42 or great cardiac vein 44. A pulse generator which is part of the electronic circuit 15 enclosed in the implantable housing 13 is connected to and couples the first pair of electrodes 51 (50, 52) via leads 20, 24. Thus, a first pair of electrodes 51 thereby provides a first current pathway 53 for delivering a first defibrillation pulse therebetween.

A second pair of electrodes are disposed to provide a second current pathway 55 different from the first current pathway 53. As illustrated in FIG. 2, a second pair consisting of a third electrode 58 and a fourth electrode 60. As illustrated, the third electrode 58 may be configured for positioning in the proximal coronary sinus (PCS) 42 and the fourth electrode 60 is configured for positioning in the left pulmonary artery (LPA) 45. A second pulse may then be generated by the pulse generator and delivered via leads 20, 24 to be delivered by the, electrically coupled or paired PCS and LPA electrodes 58, 60.

As illustrated in FIG. 2, the first lead 20 in this embodiment comprises an endocardial transvenous elongate lead having electrodes 52 and 58 arranged for establishing electrical communication with the opposing (or paired) electrode as described hereinabove. The lead is typically enclosed in a catheter and flexibly arranged to be fed through the superior vena cava 48, into the right atrium 50, and then into the coronary sinusous 40 and advanced into the coronary sinus so that the distal electrode 52 in the distal coronary sinus (DCS) may be within the CS adjacent the LV 34 and beneath the LA, or within the great cardiac vein 44 adjacent the left ventricle 34. The proximal CS electrode (described in this embodiment as the third electrode, supra) 58 is spaced apart from the DCS electrode such that when the DCS electrode is properly positioned, the PCS electrode remains in the CS.

The second lead 24 in FIG. 2 comprises an endocardial transvenous elongate lead having electrodes 50 and 60 arranged for establishing electrical communication with an opposing or paired electrode to provide an appropriate shock pulse therebetween. As such, the lead is typically enclosed in a catheter and flexibly arranged to be fed into the superior vena cava 48 and advanced into the pulmonary artery 45. The fourth electrode or pulmonary artery electrode is preferably on the tip or tip portion of the lead 24 so as to be insertably positioned in the pulmonary artery adjacent the LA 38. Upon proper placement, the first electrode or PA electrode 50 is spaced on the lead apart from the LPA electrode 60 so that when in place it is positioned in the PA 36 or superior vena cava 48.

Also illustrated in FIG. 2 is a fifth electrode 65 configured for positioning in the apex portion of the RV 32. This electrode is typically a monitoring electrode for providing the selected cardiac wave signals, i.e., P, R, or T, via associated lead line 28 to a synchronization monitor 72 and a controller in the electronic circuit 15 which allows the controller 74 to synchronize the delivery of the stimulation pulses in response to the detected cardiac or ventricular signals to minimize the possibility of induced ventricular fibrillation.

As previously stated, any of these electrodes may be hooked up to a drug delivery system. These systems include osmotic pumps, drug reservoirs and other drug delivery systems known in the art.

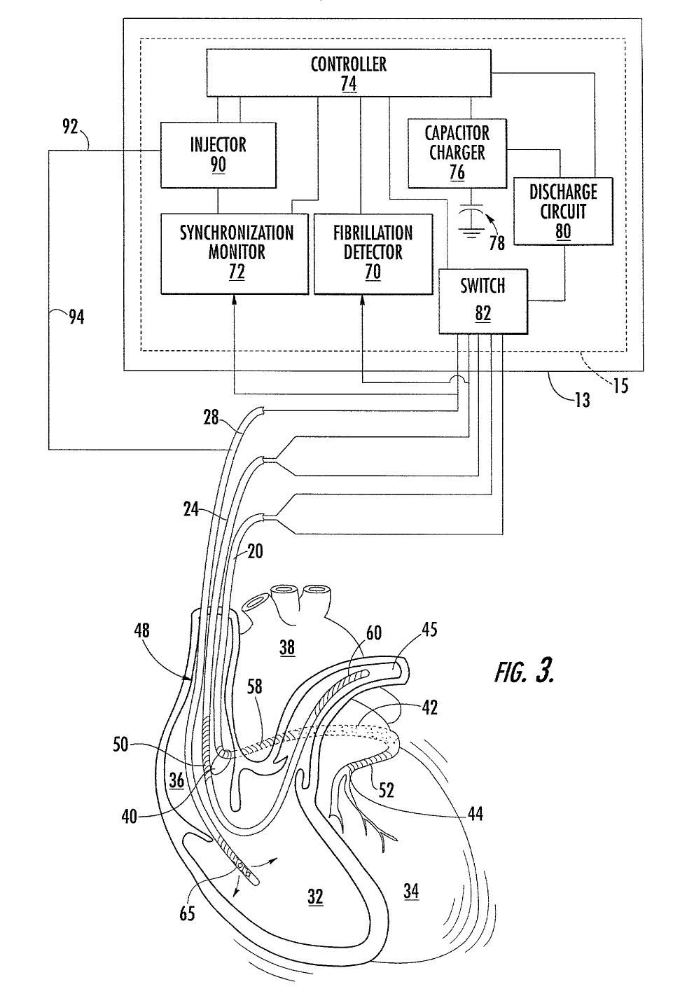

FIG. 3 illustrates an example of an implantable housing 13 containing an electronic circuit 15, which includes one or more amplifiers (not shown) for amplifying sensed cardiac signals. The amplified signals are analyzed by a fibrillation detector 70 which determines if fibrillation or an arrhythmia is present. The fibrillation detector 70 may be one of several known to those skilled in the art. Although, as illustrated, a sensing signal is provided by the RA electrode 50, it will be appreciated by those of skill in the art that the sensing electrode may also be a plurality of sensing electrodes with a plurality of signals, such as bipolar configurations, and may also be electrodes that are positioned in alternate cardiac areas as is known in the art, such as for example, the CS. In addition, the input line to the fibrillation detector may be a plurality of lines which if providing only sensing will provide an input to the detector.

The defibrillation electrodes may alternately be configured to sense cardiac cycles, or may have smaller sensing electrodes placed adjacent thereto and thereby provide input to the electronics package as well as provide a predetermined stimulation shock output to predetermined cardiac areas as directed by the controller. The controller may also direct the amount of calcium control blocker, calmodulin blocker, calmodulin kinase inhibitor or antiarrhythmic drug to be inserted into a subject based upon the occurrence of an arrhythmia.

The electronic circuit 15 also includes a cardiac cycle monitor (“synchronization monitor 72”) for providing synchronization information to the controller 74. Upon a signal from the fibrillation detector 70, the controller 74, in turn, signals a capacitor charging circuit 76 which then charges the storage capacitor 78 to a predetermined voltage, typically from a battery source (not shown). The discharge of the capacitor is controlled by the controller 74 and/or a discharge circuit 80. The controller, based on information from the synchronization monitor 72, typically allows or directs the preselected shock pulse to be relayed to either a discharge circuit for further processing (i.e., to further shape the waveform signal, time the pulse, etc.) or directly to a switch. The controller also typically controls the proper selection of the predetermined defibrillation electrode pair(s) to direct the switch to electrically activate a desired electrode pair to align the predetermined electric shock pulse pathway through which the shock pulse is provided. Additionally, the controller 74 may signal that the device containing the calcium channel blocker, calmodulin blocker, calmodulin kinase inhibitor or antiarrhythmic drug is low and an infusion of the therapeutic agent is required to maintain the system.

Therefore, it will be appreciated by those of skill in the art that the capacitor 78 may be a single capacitor or a bank of parallel capacitors sufficiently charged and sized to be able to provide at least two separate shock pulses to predetermined electrodes positioned in the heart. Additionally, the capacitor 78 can be two or more separately charged capacitors (or bank of parallel capacitors) on separate lines to provide two separate and sequential shock pulses as controlled by the controller 74 and/or the discharge circuit 80. However, it is preferred that the capacitor 78 be a relatively large capacitor for insuring sufficient charge and decay period (i.e., long time constant and low tilt) to provide sufficient energy for two shock pulses. For example, a capacitor with capacitance in the range of 200-1000 μf or more, having an associated time constant in the range of 30 ms, would typically be charged to approximately 100-200 volts and would deliver a V (peak) in a typical first waveform of about 50-100 volts leading edge. If additional shocks beyond two are administered, then a larger capacitor may be employed. In the alternative wherein the electronic package employs a circuit to further shape the waveform, the capacitor may be charged to a higher voltage range (such as around 200 V).

In FIG. 3, the pulse generator may include a single capacitor 78, and the controller 74 includes a switch (e.g., a crosspoint switch) operatively associated with that capacitor. The switch is configured to provide a biphasic pulse (i.e., a first phase of a pulse of a predetermined polarity followed by a second phase of a pulse of reversed polarity) as the first atrial defibrillation pulse and a biphasic pulse as the second atrial defibrillation pulse.

The controller 74 delivers a preselected electrical pulse to predetermined electrode pairs through a switch 82 which is preferably programmable. The capacitor charger 76, capacitor 78, controller 74, discharge circuit 80 and switch 82 thus form an electrical pulse generator. Therefore, it will be appreciated that in operation, in response to an input from the fibrillation detector 70, the controller 74 controls the pulse generator to synchronize the delivery of the timed pulse output to the proper electrode pair in accordance with the cardiac cycle information received from the synchronization monitor 72 and the specific electrode configuration employed by the device. Further, when employing a biphasic waveform, it will be appreciated by those of skill in the art that the pulse generator also includes a crosspoint switch to switch the polarity of the electrode pair for delivery of the second (inverted or negative) waveform phase. It is also preferable that the electronic package includes a receiver/transmitter coupled to the internal controller 74 for communicating with an external controller. Thus the pulse regimen could be altered by external input to the controller to alter for example, the waveform, the voltage, the electrode coupling, or even to retrieve data monitoring data received and stored in memory about the number of fibrillation episodes and the effectiveness of the shock level.

The controller 74 may also signal a drug delivery system which may include an osmotic pump located at one end of a catheter to drive therapeutic agent into the heart tissue or bloodstream via the catheter or by using a needle. A hollow helix fluid transport system can be employed as known in the art. The calcium channel blocker, calmodulin blocker, calmodulin kinase inhibitor or antiarrhythmic drug may be delivered via a fluid pathway system and into a drug reservoir. The reservoir may be loaded before, during, or after implantation from the proximal end of the drug delivery catheter. Once advanced into the heart tissue, diffusion of the liquids across the semipermeable membrane may occur because of an osmotic salt. The calcium channel blockers may be advanced into the heart or bloodstream at any time during a fibrillation or arrhythmia, but preferably before and defibrillation threshold shock.

Placing an osmotic pump directly at the site where agents are delivered has the benefit of limiting the amount of agent in the system. In devices where the agent in the filling tube can be removed, the site-specific osmotic pump does not require a long length of tubing filled with the aforementioned pharmacological agent. To deliver agents by a fluid pathway along the length of a catheter system requires a length of tubing to be filled with the appropriate agent. Although minimizing the cross sectional area of such a tube reduces excessive amounts of agents, putting the pump at the site for delivery eliminates the problem. Placing the osmotic device at the end of the catheter tube provides the advantageous means for follow-up delivery after the pump has delivered all of the agents in the reservoir. Further, only a very small amount of agent is required and the osmotic pump is placed on a catheter at the site for delivery. Additionally, the catheter may comprise a mechanism to slowly elute the therapeutic drug.

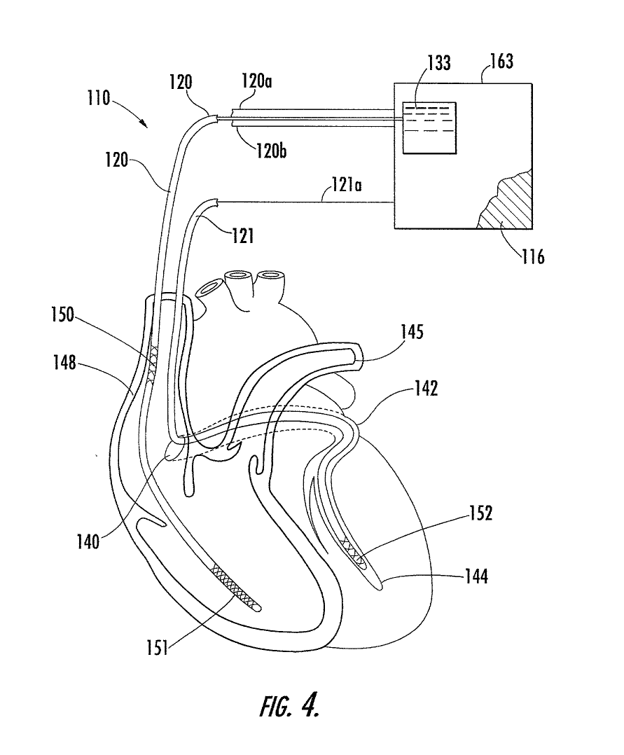

Alternatively, implantable pumps such as those used for administering insulin as well as other chemical compounds may be used as known in the art. These pumps may employ an implantable sensor and drug delivery pump system. These systems are known to incorporate sensors to perform analysis of blood as well as electrical signals from a subject's heart in order to know at what time period chemical reagents are injected into a subject's body. Typically, these reagents periodically need to be replenished, which imposes the requirement of access below the surface of the skin through which fresh reagents must be injected from time to time. FIG. 4 depicts possible embodiments of the invention. Specifically, an injector 90 is shown as part of the control system. A calcium control blocker, calmodulin blocker, calmodulin kinase inhibitor or antiarrhythmic drug may be placed into the injector when the device is produced, or through a wire 92 that can be connected to a system outside of a subject's body. Alternatively, a catheter 94 may be used to inject the calcium channel blocker or drug directly into the bloodstream rather than through the device.

In this potential embodiment, the switch 82 is programmable (e.g., by remote control such as by a radio signal) to alter the coupling of the pulse generator to the defibrillation electrodes. This feature is advantageously employed when multiple electrodes are implanted so that the electrode pairs that deliver the first and second defibrillation pulses may be changed to optimize the technique for a particular patient as well as for adding a therapeutic agent during defibrillation shock.

The energy of the first defibrillation pulse for an atrial defibrillation is preferably not greater than 8 joules, more preferably not greater than 6 joules, still more preferably not greater than 4 joules, and most preferably not greater than 2 joules. The energy of the second defibrillation pulse is typically not greater than the energy of the first defibrillation pulse (although such a result is possible where a dual capacitor design is employed), and is preferably not greater than 8 joules, more preferably not greater than 6 joules, still more preferably not greater than 4 joules, and most preferably not greater than 2 joules. The second defibrillation pulse preferably follows the first defibrillation pulse by 0 to 500 milliseconds, and more preferably follows the first defibrillation pulse by 0 to 200 milliseconds. In the alternative, the second defibrillation pulse may overlap the first defibrillation pulse, for example by from one fourth to three fourths of the total shock duration (the duration of both shocks in series). The duration of each shock may be, for example, from three to twenty milliseconds, with total shock duration being, for example, from four and one half to forty milliseconds. The energy of the defibrillation pulses for a ventricular arrhythmia are preferably not greater than 34 joules, and more preferably not greater than 27 joules.

As an alternative to a defibrillation detector, the defibrillation pulses may be triggered by an external signal administered by a technician, with the technician monitoring the patient for the appropriate time of administration. The technician may also administer the calcium channel blocker, calmodulin blocker, calmodulin kinase inhibitor or antiarrhythmic drug intravenously through an injection by a needle.

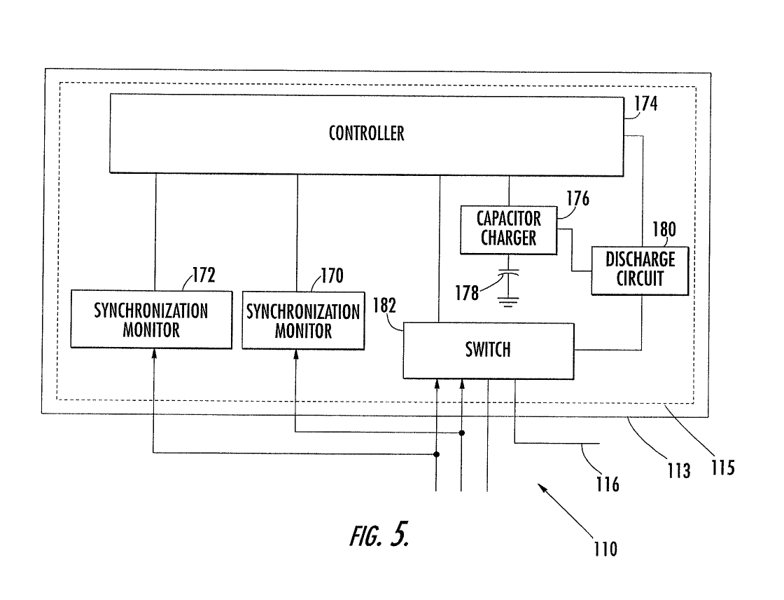

Another embodiment of a defibrillator is illustrated in FIG. 4. FIG. 4 depicts implantable housing 113 that may contain a hermetically sealed electronic circuit 115 (see FIG. 5). The housing includes an electrode comprising an active external portion 116 of the housing, with the housing 113 preferably implanted in the left or right thoracic region of the patient (e.g., subcutaneously or submuscularly, in the left or right pectoral region, or subcutaneously or submuscularly in the left or right (preferably left) abdominal region; the left pectoral region is most preferred) in accordance with known techniques as described in G. Bardy, U.S. Pat. No. 5,292,338.

The system includes a first catheter 120 and a second catheter 121, both of which are insertable into the heart (typically through the superior or inferior vena cava) without the need for surgical incision into the heart. Each of the catheters 120, 121 contains electrode leads 120a, 120b, 121a, respectively. As illustrated in FIG. 4, the system includes an electrode A; 150 that resides in the superior vena cava or innominate vein, an electrode B; 151 positioned in the right ventricle, and an electrode C; 152 positioned within a vein on the postero lateral surface of the left ventricle (e.g., in the apical third of the posterior cardiac vein or the apical half of the great cardiac vein). The active external portion of the housing 16 serves as a fourth electrode D. Designations “A” through “D” herein refer to electrodes in the aforesaid positions. The catheters may also be attached to any variety of drug delivery systems contained within the implantable housing 113.

Electrode C may be a hollow electrode to allow the flow of blood through the electrode (e.g., a stent-type electrode that engages the vessel wall) when positioned in the vein, or may be a solid electrode configured (that is, of a shape and size) to allow the flow of blood around the electrode when positioned within the vein. Electrode C may be positioned entirely within a vein on the postero-lateral surface of the left ventricle, or may also extend into the coronary sinus (as in the case of an elongate electrode).

FIG. 5 further illustrates the embodiment discussed in FIG. 4 as an example of an implantable housing 113 containing an electronic circuit 115, which includes one or more amplifiers (not shown) for amplifying sensed cardiac signals. The amplified signals are analyzed by a detector 170 which determines if ventricular fibrillation (or other arrythmia, depending on the specific treatment for which the device is configured) is present. The detector 170 may be one of several known to those skilled in the art. Although, as illustrated, a sensing signal is provided by the electrode A 150, it will be appreciated by those of skill in the art that the sensing electrode may also be a plurality of sensing electrodes with a plurality of signals, such as bipolar configurations, and may also be electrodes that are positioned in alternate cardiac areas as is known in the art, such as for example, the CS. In this situation, the input line to the detector may be a plurality of lines which if providing only sensing will provide an input to the detector.

The defibrillation electrodes may alternately be configured to sense cardiac cycles, or may have smaller sensing electrodes placed adjacent thereto and thereby provide input to the electronics package as well as provide a predetermined stimulation shock output to predetermined cardiac areas as directed by the controller.

The electronic circuit 115 also includes a cardiac cycle monitor (“synchronization monitor 172”) for providing synchronization information to the controller 174. As discussed below, the synchronization is typically provided by sensing cardiac activity in the RV, but may also include other sensing electrodes which can be combined with the defibrillation electrodes or employed separately to provide additional assurance that defibrillation shock pulses are not delivered during sensitive portions of the cardiac cycle so as to reduce the possibility of inducing ventricular fibrillation. Controller 174 also senses when drug delivery is needed and provides the appropriate electrical response for the drug delivery system to operate.

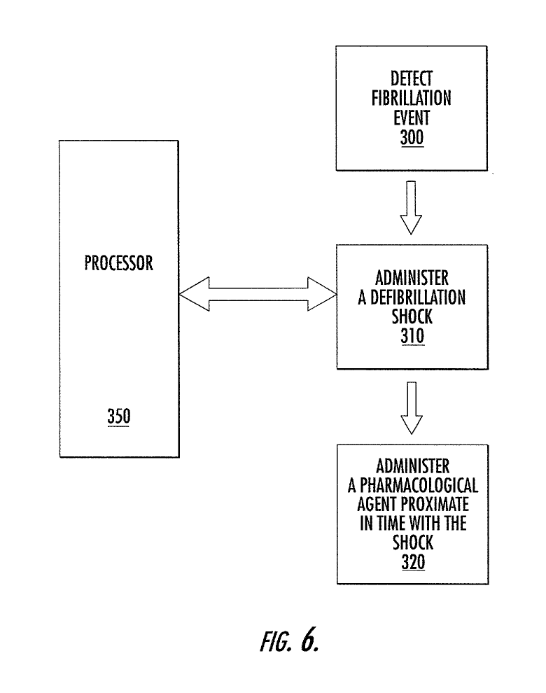

FIG. 6 is a block diagram of a method for controlling the delivery of defibrillation shocks according to the embodiments of the present invention. Thus, a system may be configured to include a processor 350 that detects a fibrillation event 310. The processor 350 may also signal a defibrillation source, wherein a therapeutic shock is administered 320 after the processor detects the fibrillation event. Additionally, the processor may determine the level of shock to be produced. The therapeutic shocks may also be accompanied by a therapeutic drug proximate in time to the therapeutic shock. The therapeutic drug may be received from a drug reservoir comprising a therapeutic drug selected from the group consisting of a calcium channel blocker, a calmodulin blocker and a calmodulin kinase inhibitor, wherein the processor 350 is configured to determine the amount of the therapeutic drug to be administered.

Numerous configurations of capacitor and control circuitry may be employed. The power supply may include a single capacitor, and the control circuit may be configured so that both the auxiliary pulse and the defibrillation pulse are generated by the discharge of the single capacitor. The power supply may include a first and second capacitor, with the control circuit configured so that the auxiliary pulse is generated by the discharge of the first capacitor and the defibrillation pulse is generated by the discharge of the second capacitor. In still another embodiment, the power supply includes a first and second capacitor, and the control circuit may be configured so that the auxiliary pulse is generated by the discharge (simultaneous or sequential) of both the first and second capacitors, and the defibrillation pulse likewise generated by the discharge of the first and second capacitors. The controller's power supply may include a 20 to 400 microfared capacitor. Other cardiac systems known in the art may also be used as a part of this invention.

In one embodiment of the invention, a subject afflicted with an abnormal cardiac cycle as described herein is administered a therapeutically-effective amount of the compound of a calcium channel blocker, calmodulin blocker, calmodulin kinase inhibitor, antiarrhythmic drug, or a pharmaceutically acceptable salt thereof. A “therapeuticallyeffective” amount as used herein is an amount of a calcium channel blocker or drug that is sufficient to alleviate (e.g., mitigate, decrease, reduce) the defibrillation threshold. It is not necessary that the administration of the compound eliminate the defibrillation threshold, as long as the benefits of administration of compound outweigh the detriments. Likewise, the terms “treat” and “treating”, as used herein, are not intended to mean that the subject is necessarily cured, only that some alleviation or improvement in the condition of the subject is effected by administration of the compound.

Suitable subjects of the present invention include humans and animals. When the subject is an animal, mammals are preferred, with livestock and primates being particularly preferred. Humans are the most preferred subjects. Subjects may be adult, adolescent, juvenile, infant, or neonatal.

Subjects may be administered the compounds and compositions of the present invention by any suitable means. Exemplary means are parenteral administration (including subcutaneous, intradermal, intramuscular, intravenous and intraarticular). The formulations may conveniently be presented in unit dosage form and may be prepared by any of the methods well known in the art.

In the manufacture of a medicament according to the invention (the “formulation”), active compounds or the pharmaceutically acceptable salts thereof (the “active compounds”) are typically admixed with, inter alia, an acceptable carrier. The carrier must, of course, be acceptable in the sense of being compatible with any other ingredients in the formulation and must not be deleterious to the patient. One or more active compounds may be incorporated in the formulations of the invention, which formulations may be prepared by any of the well known techniques of pharmacy consisting essentially of admixing the components, optionally including one or more accessory therapeutic ingredients.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets. The compound or salt may be provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable, may be employed in sufficient quantity to emulsify the compound or salt in an aqueous carrier.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes produced may be reduced in size through the use of standard sonication and homogenization techniques or other techniques known in the art.

Of course, the liposomal formulations containing the pharmaceutically active compounds identified with the methods described herein may be lyophilized to produce a lyophilizate which may be reconstituted with a pharmaceutically acceptable carrier, such as water, to regenerate a liposomal suspension.

In addition to the active compounds, the pharmaceutical formulations may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical formulations of the present invention may be lyophilized using techniques well known in the art.

Pharmaceutical formulations of the present invention may comprise compounds of the present invention in lyophilized form. Alternatively, pharmaceutical formulations of the present invention may comprise compounds of the present invention in a pharmaceutically acceptable carrier. Such pharmaceutical formulations are generally made by admixing the compounds described herein with a pharmaceutically acceptable carrier. Pharmaceutically acceptable carriers are preferably liquid, particularly aqueous, carriers, the selection of which are known in the art. For the purpose of preparing such formulations, the compound may be mixed in a buffered saline (e.g., pH 6 to 8) or conventional culture media. The formulation may be stored in a sterile glass container sealed with a rubber stopper through which liquids may be injected and formulation withdrawn by syringe.

With respect to all the methods described herein, a therapeutically effective dosage of any specific compound, the use of which is in the scope of present invention, may vary somewhat from compound to compound and subject to subject, and will depend upon the condition of the subject and the route of delivery. A dosage from about 1 mg/kg/hr to about 10 mg/kg/hr of subject body weight, or preferably about 4 mg/kg of subject body weight, administration.

The concentration of the compound of the present invention or a pharmaceutically acceptable salt thereof in a formulation of the present invention may be determined by the skilled artisan and will vary according to certain conditions, including the characteristics of subject being treated (e.g., species, age, weight), the dosage form being used, and the like.

The compounds of the present invention may be administered in conjunction with other compounds, as may be determined by the skilled artisan.

The present invention is explained in greater detail in the Materials and Methods section. These examples are intended as illustrative of the invention, and are not to be taken as limiting thereof.

Materials and Methods

Animal Preparation and Electrode Placement

Six healthy pigs (30-35 kg) of either sex were anesthetized, monitored, and maintained under physiologic conditions. See, Chattipakorn et al. (2000). Two catheter-mounted platinum coated titanium coil electrodes were used as defibrillating electrodes. A 34-mm catheter was inserted into the right ventricular (RV) apex and served as the cathode for the first phase of the biphasic shock. A 68-mm catheter was positioned at the junction of the superior vena cava (SVC) and right atrium. The position of the catheters was verified with fluoroscopy. The blood pressure and heart rate were monitored continuously throughout the entire study.

Defibrillation Protocol

Ventricular fibrillation was induced by 60-Hz alternating current delivered via an electrode at the tip of the RV catheter. After 10 seconds of ventricular fibrillation, defibrillation was attempted with 10-ms biphasic truncated exponential shocks. Delivered voltage and current were recorded on a waveform analyzer and total delivered energy was calculated. A minimum of 4 min was allowed to elapse between ventricular fibrillation episodes. If the shock failed to defibrillate, a rescue shock (20-30 J) was delivered within 10 sec.

The defibrillation threshold was determined 4 times during 4 different conditions in this study. The defibrillation threshold in each group was determined using a 3-reversal up/down protocol. See Chattipakorn et al. The first defibrillation threshold was determined at the beginning of the study. This defibrillation threshold was used as a control defibrillation threshold (control-DFT). Then, Flunarizine, a delayed afterdepolarization inhibitor, was injected intravenously as a bolus (2 mg/kg) and maintenance (4 mg/kg/hr) dose. The second defibrillation threshold (drug-DFT) was determined 15 minutes after the application of Flunarizine. After the drug-DFT was obtained, the administration of Flunarizine was terminated while the blood pressure and heart rate were monitored. The third defibrillation threshold (washout-DFT) was determined again when the blood pressure returned to the control level which normally took 60 minutes after the termination of the drug. To confirm that cyclodextrin (a vehicle used in the Flunarizine preparation) did not alter the defibrillation threshold, cyclodextrin of the same volume as used in the Flunarizine injection was injected intravenously and the fourth defibrillation threshold was determined (vehicle-DFT).

Statistical Analysis

Values are shown as mean standard deviation. Comparisons of data among different defibrillation threshold groups were performed using an analysis of variance. When statistical significance was found, individual differences were analyzed with a Fisher's post-hoc test. Differences were considered significant when P<0.05.

Results

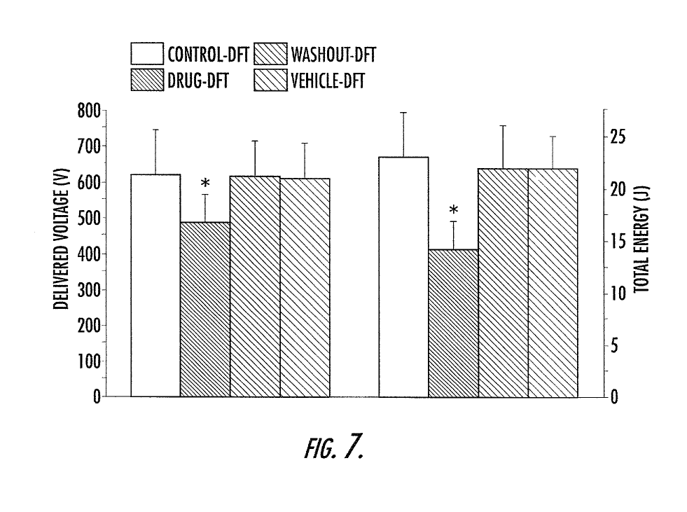

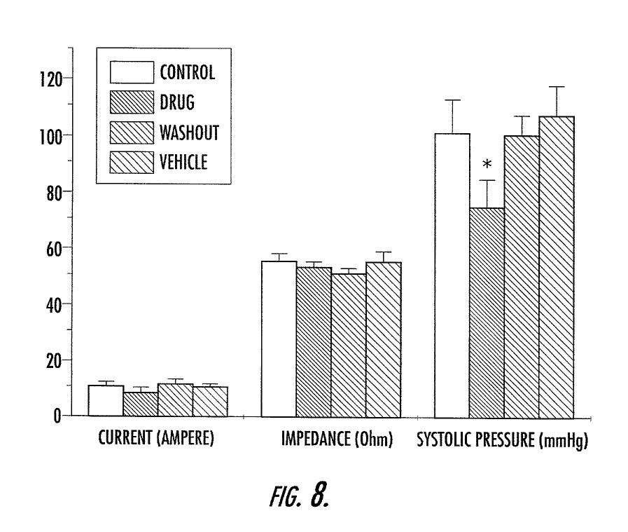

The delivered leading edge voltage and energy for the control-DFT was 623±120 volts and 23±4 joules, respectively. The vehicle did not alter the defibrillation threshold (606±96 volts, 22±3 joules, FIG. 7). The drug-DFT (485±82 volts, 14±3 joules) was significantly lower than the control-DFT. The washout defibrillation threshold was not significantly different from the control-DFT (FIG. 7). Flunarizine application significantly reduced the defibrillation threshold by approximately 22% by leading edge voltage and by approximately 40% by energy. FIG. 7 also illustrates a graphical depiction of the delivered leading edge voltage and the total energy for the defibrillation threshold. The delivered voltage and total energy for the drug-defibrillation threshold was significantly lower than the control-defibrillation threshold. The washout-defibrillation threshold was neither different from the control-defibrillation threshold nor the vehicle-defibrillation threshold. (The * indicates P<0.01 vs. control-defibrillation threshold, washout-defibrillation threshold, and vehicle-defibrillation threshold.) There were no significant changes in the current or impedance among the four defibrillation threshold groups (FIG. 8). Flunarizine, however, significantly reduced the systolic blood pressure by ˜26% (76±10 mmHg vs. 102±11 mmHg for a control). FIG. 8 also illustrates in graphical format the current, impedance, and systolic blood pressure in four measured groups. There was no difference among the four groups for the current and the impedance during the defibrillation threshold determination. The systolic blood pressure was significantly lower after Flunarizine was injected compared to the pressure during the control, vehicle injection, and after the drug washout. The * indicates P<0.01 vs. control, washout, and vehicle.

The foregoing examples are illustrative of the present invention, and are not to be construed as limiting thereof. The invention is defined by the following claims, with equivalents of the claims to be included therein.

What is claimed is:

1. A method of decreasing the shock strength needed to treat an arrhythmia comprising:
detecting an arrhythmia in the heart of a subject;
administering a therapeutic electric shock to the heart of said subject to treat said arrhythmia; and
administering a therapeutic drug selected from the group consisting of a calcium channel blocker, a calmodulin blocker, a calmodulin kinase inhibitor and an antiarrhythmic drug to said subject at a time and in an amount effective to decrease the strength of said shock required to treat said arrhythmia.

2. The method according to claim 1 wherein said calcium channel blocker is selected from the group consisting of amiodarone, bepridil, D600, diltiazem, felodipine, flunarizine, isradipine, nicardipine, nifedipine, nimodipine and verapamil.

3. The method according to claim 1 wherein said antiarrhythmic drug is selected from the group consisting of adenosine, aprindine, doxorubicin, ryanodine, ethmozin, dofetilide and ibutilide.

4. The method according to claim 1 wherein said calmodulin blocker is a calmodulin kinase inhibitor.

5. The method according to claim 1, wherein said arrhythmia is an atrial arrhythmia.

6. The method according to claim 1, wherein said arrhythmia is a ventricular arrhythmia.

7. The method according to claim 1, wherein said therapeutic electric shock is not greater than 34 joules.

8. The method according to claim 1, wherein said calcium channel blocker is administered in an amount effective to reduce defibrillation threshold shock in said subject by at least 10% as compared to a normal defibrillation threshold shock.

9. The method according to claim 8, wherein said defibrillation threshold shock is reduced by at least 20% in leading edge voltage.

10. The method according to claim 8, wherein said defibrillation threshold shock is reduced by at least 40% in energy.

11. The method according to claim 1, wherein said calcium channel blocker is administered in an amount effective to inhibit a delayed afterdepolarization caused by said shock in the absence of said calcium channel blocker.

12. A cardiac device for treating arrhythmia in a subject in need thereof, comprising:
an arrhythmia detector configured to determine if a cardiac arrhythmia is occurring from electrical activity sensed from the heart of a subject;
a controller operatively associated with said detector;
an injector operatively associated with said controller to administer a therapeutic drug selected from the group consisting of calcium channel blocker, a calmodulin blocker, a calmodulin kinase inhibitor and an antiarrhythmic drug to said subject when a cardiac arrhythmia is detected; and
a shock generator operatively associated with said controller and configured to deliver a therapeutic electric shock to the heart of said subject.

13. The cardiac device of claim 12 further comprising said controller configured to deliver said therapeutic drug at said time with said therapeutic electric shock so that the strength of said shock is decreased as compared to the shock required to treat said arrhythmia in the absence of administration of said therapeutic drug.

14. The cardiac device of claim 12 wherein said calcium channel blocker is selected from the group consisting of amiodarone, bepridil, D600, diltiazem, felodipine, flunarizine, isradipine, nicardipine, nifedipine, nimodipine and verapamil.

15. The cardiac device of claim 12 wherein said antiarrhythmic drug is selected from the group consisting of adenosine, aprindine, doxorubicin, ryanodine, ethmozin, dofetilide and ibutilide.

16. The cardiac device of claim 12 wherein said calmodulin blockers is a CaM kinase.

17. The cardiac device of claim 12 wherein said detector is an atrial arrhythmia detector.

18. The cardiac device of claim 12 wherein said detector is a ventricular arrhythmia detector.

19. The cardiac device of claim 12 wherein said device is an internal device.

20. The cardiac device of claim 12 wherein said device is an external device.

21. The cardiac device of claim 12 wherein said therapeutic electric shock is not greater than 34 joules.

22. A cardiac device comprising:
a sensing electrode positioned within a subject's heart configured to measure signals from said subject's heart;
a power supply;
a controller operatively associated with said sensing electrode;
a plurality of electrodes operatively associated with said controller configured to deliver a therapeutic electric shock to said subject's heart;
a catheter operatively associated with said controller configured to deliver a therapeutic amount of a therapeutic drug selected from the group consisting of calcium channel blocker, a calmodulin blocker, a calmodulin kinase inhibitor and an antiarrhythmic drug to said subject.

23. The cardiac device of claim 22 wherein said plurality of electrodes configured for sensing cardiac signals determine if a subject's heart is undergoing an arrhythmia or a fibrillation.

24. The cardiac device of claim 22 wherein said calcium channel blocker is selected from the group consisting of amiodarone, bepridil, D600, diltiazem, felodipine, flunarizine, isradipine, nicardipine, nifedipine, nimodipine and verapamil.

25. The cardiac device of claim 22 wherein said antiarrhythmic drug is selected from the group consisting of adenosine, aprindine, doxorubicin, ryanodine, ethmozin, dofetilide and ibutilide.

26. The cardiac device of claim 22 wherein said calmodulin blockers is a CaM kinase.

27. The cardiac device of claim 22 wherein said device is internal.

28. The cardiac device of claim 22 wherein said device is external.

29. A method of decreasing the shock strength needed to treat an arrhythmia comprising:
detecting an arrhythmia in the heart of a subject;
administering a therapeutic electric shock to the heart of said subject to treat said arrhythmia;
administering a therapeutic drug selected from the group consisting of a calcium channel blocker, a calmodulin blocker, a calmodulin kinase inhibitor and a first antiarrhythmic drug to said subject at a time and in an amount effective to decrease the strength of said shock required to treat delayed afterdepolarizations; and
administering a second antiarrhythmic drug at a time and in an amount effective to prolong the refractory period.

30. The method according to claim 29 wherein said calcium channel blocker is selected from the group consisting of amiodarone, bepridil, D600, diltiazem, felodipine, flunarizine, isradipine, nicardipine, nifedipine, nimodipine and verapamil.

31. The method according to claim 29 wherein said first antiarrhythmic drug is selected from the group consisting of adenosine, aprindine, doxorubicin, ryanodine, and ethmozin

32. The method according to claim 29 wherein said second antiarrhythmic drug is selected from the group consisting of dofetilide and ibutilide.

33. The method according to claim 29 wherein said calmodulin blockers is a CaM kinase.

34. A cardiac device for treating arrhythmia in a subject in need thereof, comprising:
an arrhythmia detector configured to determine if a cardiac arrhythmia is occurring from electrical activity sensed from the heart of a subject;
a controller operatively associated with said detector;
an injector operatively associated with said controller to administer a therapeutic drug selected from the group consisting of calcium channel blocker, a calmodulin blocker, a calmodulin kinase inhibitor and a first antiarrhythmic drug to said subject when a cardiac arrhythmia is detected, wherein said injector operatively associated with said controller administers a second antiarrhythmic drug; and
a shock generator operatively associated with said controller and configured to deliver a therapeutic electric shock to the heart of said subject.

35. The cardiac device of claim 34 wherein said calcium channel blocker is selected from the group consisting of amiodarone, bepridil, D600, diltiazem, felodipine, flunarizine, isradipine, nicardipine, nifedipine, nimodipine and verapamil.

36. The cardiac device of claim 34 wherein said first antiarrhythmic drug is selected from the group consisting of adenosine, aprindine, doxorubicin, ryanodine, and ethmozin

37. The cardiac device of claim 34 wherein said second antiarrhythmic drug is selected from the group consisting of dofetilide and ibutilide.

38. The cardiac device of claim 34 wherein said calmodulin blockers is a CaM kinase.

39. The cardiac device of claim 34 wherein said device is an internal device.

40. The cardiac device of claim 34 wherein said device is an external device.

41. A system for treating an arrhythmia comprising:
a processor to detect a fibrillation event;
a defibrillation source, wherein a therapeutic shock is administered after the processor detects the fibrillation event;
a drug reservoir comprising a therapeutic drug selected from the group consisting of a calcium channel blocker, a colmodulin blocker and a calmodulin kinase inhibitor, wherein the therapeutic drug is administered proximate in time with the therapeutic shock and wherein the processor is configured to determine the amount of the therapeutic drug to be administered and the level of shock to be produced.

42. The system according to claim 41, wherein the fibrillation event is an atrial arrhythmia event.

43. The system according to claim 41, wherein the fibrillation event is a ventricular arrhythmia event.

44. The system according to claim 41 wherein said calcium channel blocker is selected from the group consisting of amiodarone, bepridil, D600, diltiazem, felodipine, flunarizine, isradipine, nicardipine, nifedipine, nimodipine and verapamil.

45. The system according to claim 41 wherein said antiarrhythmic drug is selected from the group consisting of adenosine, aprindine, doxorubicin, ryanodine, ethmozin, dofetilide and ibutilide.

46. The system according to claim 41 wherein said calmodulin blockers is a CaM kinase.

47. A cardiac device for treating an arrhythmia in a subject in need thereof, comprising:
an arrhythmia detector configured to determine if the subject is likely to experience an arrhythmia;
a controller operatively associated with said detector;
an injector operatively associated with said controller to administer a therapeutic drug, said therapeutic drug selected from the group consisting of calcium channel blocker, a calmodulin blocker, a calmodulin kinase inhibitor and an antiarrhythmic drug to said subject.

48. The cardiac device of claim 34 wherein said calcium channel blocker is selected from the group consisting of amiodarone, bepridil, D600, diltiazem, felodipine, flunarizine, isradipine, nicardipine, nifedipine, nimodipine and verapamil.
